(19) United States
(12) Patent Application Publication
(10) Pub. No.: US 2002/0115904 A1
(43) Pub. Date: Aug. 22, 2002

(54) Magnetically controllable embolic materials

(76) Inventor: Brooke Ren, Champlin, MN (US)

Correspondence Address: Bryan K. Wheelock, Harness, Dickey & Pierce, Suite 400, 7700 Bonhomme, St. Louis, MO, 63105, US

(21) Appl. No.: 09/790,219

(22) Filed: Feb. 21, 2001

(51) Int. Cl. A61N 2/00

(52) U.S. Cl. 600/12

(57) ABSTRACT

A settable, magnetically guideable embolic material for occluding vascular defects, the material comprising: prolamine, ethanol, a magnetically responsive material, and a radio-opaque material.

### FIELD OF THE INVENTION

This invention relates to embolic materials, and in particular to magnetically controllable magnetic materials.

### BACKGROUND OF THE INVENTION

The artificial blocking of blood flow is often called “embolization”. There are a number of reasons why it may be desirable to block a blood vessel, for example to treat a defect in vessel, such as a aneurysm or a arterial-venous malformation (AVM); to restrict blood flow to malfunctioning portions of an organ or to a tumor; and to control bleeding induced by trauma or during surgery. A variety of liquid embolic agents which can be introduced into the vasculature to embolize a selected vessel or selected vessels have been developed. Examples of the materials include that disclosed in Slaikeu, U.S. Pat. No. 6,160,025, incorporated herein by reference.

A principal difficulty with liquid embolic materials has been controlling them so that they do not inadvertently embolize other blood vessels. Magnetically controllable embolic materials have been developed, for example the materials disclosed in co-assigned U.S. patent application Ser. No. U.S. patent application Ser. No. 09/430,118, filed Oct. 29, 1999, for Methods and Apparatus for Treating Vascular Defects, U.S. patent application No. U.S. patent application ser. No. 09/430,200,filed Oct. 29, 1999, Methods Of and Compositions For Treating Vascular Defects and U.S. patent application Ser. No. U.S. patent application Ser. No. 09/271,11 8,filed Mar. 17, 1999, for Magnetic Vascular Defect Treatment System, and U.S. patent application Ser. No. 09/527,108, filed Mar. 16, 2000, for Methods Of and Compositions For Treating Vascular Defects the disclosures of which are incorporated herein by reference.

Desirable materials flow sufficiently so that they can be delivered by microcatheters, set within a reasonable time, are easy to control (magnetically or otherwise), are bio-compatible, and preferably can be visualized for example in x-ray or fluoroscopic imaging.

### SUMMARY OF THE INVENTION

The present invention is a settable, magnetically guideable embolic material for occluding a blood vessel. Generally the material comprises prolamine, polyvinyl acetate (PVAc), ethanol, a magnetically responsive material, and a radio-opaque material. More specifically, the material preferably comprises between about 6 weight percent and about 20 weight percent prolamine, between about 1 weight percent and about 10 percent weight percent PVAc, between about 16 weight percent and about 50 weight percent ethanol, between about 20 weight percent and about 60 weight percent magnetically responsive material, and between about 5 and about 35 percent of a radio-opaque material.

In the preferred embodiment the prolamine constitutes between about 8 weight percent and about 12 weight percent of the material. Also in the preferred embodiment the ethanol is present as an aqueous solution of between about 70% and about 95% ethanol. In the preferred embodiment, the magnetically responsive material comprises Fe₃O₄, but other suitable magnetically responsive materials could be used. In the preferred embodiment, the Fe₃O₄ comprises between about 35 weight percent and about 45 weight percent of the material. In the preferred embodiment, the radio-opaque material is tantalum, gold, platinum, and tungsten, and oxides of tantalum, gold, platinum, and tungsten, but other suitable radio opaque materials could be used. The radio-opaque material preferably comprises between about 10 weight percent and about 20 weight percent of the material.

The embolic material of the present invention is sufficiently flowable that it can be delivered via a microcatheter, yet is sufficiently viscous that it can be controlled in the blood vessel. The material is magnetically controllable with an external source magnet (either an electromagnet, a permanent magnet, or some combination of electromagnets and permanent magnets). The material is radio-opaque so that it can be visualized in x-rays and fluoroscopic images.

### DETAILED DESCRIPTION OF THE INVENTION

The settable, magnetically controllable embolic material of the present invention is adapted to be delivered into a patients vasculature, controlled by the application of a magnetic field from an external source magnet or magnets (electromagnets, permanent magnetic, or a combination of electromagnets and permanent magnets) for example, the magnetic disclosing in co-pending application Ser. No. 60/255,245, filed Dec. 13, 2000, and incorporated herein by reference, to set and embolize the vessel in which it is delivered. The composition is useful in treating vascular defects such as aneurysms and arterial-venous malformations (AVM's), and blocking blood flow to damaged portions of organs and to tumors. The material preferably comprises prolamine (zein), ethanol, a magnetically responsive material, and a radio-opaque material.

In the preferred embodiment, magnetically guideable embolic material for occluding vascular defects, the material comprises between about 6 weight percent and about 20 weight percent prolamine; PVAc, between about 15 weight percent and about 50 weight percent ethanol; between about 20 weight percent and about 60 weight percent magnetically responsive material; and between about 5 and about 35 percent of a radio-opaque material.

More preferably, the prolamine comprises between about 8 weight percent and about 12 weight percent prolamine, and most preferably about 10 weight percent prolamine. The prolamine has an average molecular weight of about 35,000+/−20%.

More preferably, PVAc comprises between about 1 weight percent and about 10 weight percent prolamine, and most preferably about 5 weight percent PVAc. The PVAc has an average molecular weight of about 170,000+/−20%.

More preferably, the ethanol is present as an aqueous solution of between about 70% and about 95% ethanol, it being desirable (but not necessarily essential) to avoid the use of 100% ethanol, which is highly toxic to tissue. The ethanol is most preferably in the form of an ethanol/polyethylene glycol/water solution of (85%/5%/10%). The polyethylene glycol is preferably 200 molecular weight.

More preferably, the magnetically responsive material comprises Fe₃O₄, and the embolic material is between about 35 weight percent and about 45 weight percent Fe₃O₄. The Fe₃O₄ is preferably in powder form, and can be provided as a powder coated with the radio-opaque material. The maximum particle diameter is less than about 5 microns. It is desirable that the magnetically responsive material remain suspended in the embolic material even during the application of the external magnetic field, so that the material remains magnetically controllable.

More preferably the radio-opaque material comprises at least one of tantalum, gold, platinum, and tungsten, and oxides of tantalum, gold, platinum, and tungsten, and the radio-opaque material between about 10 weight percent and about 20 weight percent radio-opaque material. The radio-opaque material allows the physician to observe the delivery of the embolic material on x-ray or fluoroscopic imaging equipment. The radio-opaque material is most preferably provided as a coating on the magnetically- responsive material, such that the maximum particle size of the coated particles is less than about 10 microns.

In addition, polyvinyl acetate may be included as a binder to insure cohesiveness of the material. The polyvinyl acetate preferably has an average molecular weight of about 170,000+/−20%.

In order to properly interact with the magnetic system the embolic must include a magnetically susceptible component (e.g., Fe₃O₄). In order to be viewed under the fluoroscope it must have a component which is radio-opaque (e.g., Au or Ta). To help insure the embolic stays in place it should have a component which adheres to the vasculature (zein). To insure the cohesiveness of the embolic it should have a binding component (PVAC). And to deliver it as a liquid it must have a liquid component that can dissolve a portion of the solids until the embolic is at the intended site at which time the liquid component is safely exchanged into the blood (Ethanol/Water mixture).

There are a number of some-times competing desirable properties for a settable, magnetically guided embolic material. First, it is desirable that the set material adheres to the surround tissue, so that embolic material does not release. Thus the embolic material desirably has adhesion properties capable of forming a bond with the vessel wall, sufficient to exceed the hydrodynamic forces typically encountered in the vasculature. The adhesion is preferably permanent. For these reasons it is desirable, but not essential, that the force necessary to delaminate the cured material from in-vivo tissue be equal to or greater than the hydrodynamic force encountered at the site.

It is also important that the cured embolic be biocompatible, i.e., that it be substantially biologically inert and not produce harmful chronic or sub chronic toxic effects, or have carcinogenic or mutagenic properties. The solvent exchanged during the curing process also should not produce harmful acute or sub-chronic toxic effects.

In addition to biocompatibility, the material must be compatible with the delivery devices and other medical equipment used in the medical procedure. These devices shall include, but may not be limited to, injectors, micro-catheters, and connectors. Compatibility refers specifically to the following: The embolic including precursor solutions should be able to successfully flow through the devices during injection. The embolic including precursor solutions should not leech any chemicals that are not biocompatible from these devices while traveling through them. The embolic including prep solutions should not damage the devices such that the patient is harmed, or the devices do not perform adequately to successfully complete the procedure.

The embolic material must be able to safely interact with at least the following materials dextrose 50%, dextrose 5%, and x-ray contrast solution. Dextrose is one possible material used to fill the lumen of the delivery catheter prior to injection of the embolic material. The embolic material will be exposed to a number of other materials during and after the procedure. These materials include contrast agent, flush agents, etc. Contact with these materials shall not change the delivery properties of the embolic material nor the materials contacted such that the outcome of the procedure remains unaffected.

The cured embolic material must be durable, i.e., it must be capable of withstanding the hydrodynamic forces typically encountered in the vasculature. The cured material is preferably at least as hard as the surrounding vasculature so that the stabilized embolic material should have sufficient mechanical integrity to resist hydrodynamic forces encountered in the vasculature. The hardness of the cured material should be sufficient to contribute to overall integrity and should not decrease significantly over the life of the implant. The compression strength of the cured material should not decrease significantly over the life of the implant. The stabilized embolic material preferably has a minimum compression resistance of between 2 lbs/in² and 10 lbs/in², and more preferably at least 5.8 lbs/in².

The minimum density of the liquid embolic material is preferably greater than the density of blood (1.057 g/cc) so it can easily be delivered through it. The maximum density of the liquid embolic shall be less than 40% of the density of platinum. Platinum coils are currently used to fill aneurysms and take up an average of 40% of the aneurysm volume. It is believed that an embolic which is less dense than this will produce less stress on the vasculature over time. The finished density of the material is preferably less than or equal to the density found in the same size defect treated with Platinum coils. Platinum coils have been approved for use for filling aneurysms. (Materials with a greater density would have to be shown not to cause vessel damage). The density of Platinum is 21.09 g/cc. The Platinum coils occupy approximately 40% of the aneurysm volume. Therefore the preferred density of fully cured, stabilized material shall be less than about 8.44 g/cc.

The liquid embolic material preferably has a magnetic susceptibility in its liquid form sufficient to allow it to be controlled within a vascular defect. The pre-delivery magnetic susceptibility needs to be strong enough that when placed in the recommended magnetic field, the force vector generated by the magnet on the embolic is greater than the gravitational force vector acting on the embolic. The magnetic force must also be greater than the force produced by normal systolic blood pressure acting on an cross sectional area equal to the average micro-catheter opening (typical diameter about 0.020 inches).

The embolic material must also have a reasonable working time, to accommodate any difficulties in the delivery procedure, yet it must set reasonable quickly so that it does not migrate after injection or during catheter removal. The injectable period shall be greater than or equal to about 5 minutes regardless of whether the injection is continuous or stopped for that period of time. Thus embolic must be able to flow through and out the catheter at a pressure of 250 psi or less to insure the integrity of the catheter. The embolic material must be able to be safely injected through a micro-catheter and remain injectable for a reasonable length of time. A setting time of approximately 30 minutes after contact with the blood is therefore desirable.

In order to insure the aneurysm has been adequately treated it is necessary for the physician to have a means see it. The liquid embolic material shall be visible to a trained physician using normal fluoroscopic equipment typically found in INR Labs.

For a minimum of 30 minutes after completion of the mix procedure the embolic must not exhibit any signs of separation visually and under x-ray.

The individual components of the liquid embolic material shall be resistant to separation by gravity over the length of time necessary to perform a standard procedure. The degree of resistance to separation by gravity shall allow a sufficient working period for the physician. The degree of resistance to magnetic separation shall be sufficient to prevent separation by the field found within the vascular defect during a standard procedure. The magnetic force is proportion to the product of the applied field and field gradient (Tesla²/m). The individual components of the liquid embolic material shall be individually resistant to separation within a magnetic field times field gradient product of 0.08 Tesla²/m or less. There shall be no separation visually present during an aneurysm injection and over the maximum catheter removal curing-period of 30 minutes.

The viscosity of the material shall be low enough to allow the embolic to be easily and safely injected through recommended micro-catheters without causing physician fatigue and at a pressure deemed safe within the catheter. Injection of the material should be easy enough to prevent fatigue of the physician's hands while at the same time providing a sense of control over the material. The viscosity of the material shall be less than or equal to 2.0 lb*s/ft² (1 N*s/m²) as determined through injection testing.

In practice a catheter is navigated to the site of the embolization. The magnetically controllable embolic material is ejected from the catheter preferably while a magnetic field is applied by an external magnet. To prevent the reactive polymer from precipitating or reacting with aqueous solution in the delivery catheter, it is preferred that the delivery microcatheter is prepped with a suitable agent, such as dextrose, contrast, glycol, fatty acid, etc. The embolic may be packaged in either two parts, or single part. In either way, the embolic should be thoroughly mixed; if it is in two parts, the embolic should be put through a static mixer at least four times to ensure the adequate mixing; if it is packaged in one part, the embolic should sit on a vortex mixer for at least two minutes prior to use.

The material tends to form in layers parallel to the local magnetic field direction, and the material is pulled in the direction of the magnetic gradient of the field. The material hardens in the vessel as the ethanol is absorbed by the blood. Preferably within about thirty minutes the material has set sufficiently that the magnetic field can be removed and the procedure ended.

What is claimed is:

1. A settable, biocompatible, magnetically guidable embolic material for occluding vascular defects, the material having: a density of between about 1 and about 8.5 grams/cc, magnetic attraction force of between about 10 and about 100 N/kg prior to setting in an applied field times field gradient product of 0.04 Tesla²/m, and having magnetic components that do not separate in a field times field gradient product of 0.08 Tesla²/m.

2. A settable, magnetically guideable embolic material for occluding vascular defects, the material comprising:
between about 6 weight percent and about 20 weight percent prolamine
between about 16 weight percent and about 50 weight percent ethanol
between about 20 weight percent and about 60 weight percent magnetically responsive material;
between about 5 and about 35 percent of a radio-opaque material.

3. The embolic material according to claim 2 further comprising polyvinyl acetate binder.

4. The embolic material according to claim 2 wherein the magnetically responsive material comprises Fe₃O₄.

5. The embolic material according to claim 4 comprising between about 35 weight percent and about 45 weight percent Fe₃O₄.

6. The embolic material according to claim 2 wherein the radio-opaque material comprises at least one of tantalum, gold, platinum, and tungsten, and oxides of tantalum, gold, platinum, and tungsten.

7. The embolic material according to claim 2 comprising between about 8 weight percent and about 12 weight percent prolamine.

8. The material according to claim 2 wherein the ethanol is present as an aqueous solution of between about 70% and about 95% ethanol.

9. The embolic material according to claim 2 comprising between about 10 weight percent and about 20 weight percent radio-opaque material.

10. The embolic material according to claim 9 wherein the radio-opaque material comprises at least one of tantalum, gold, platinum, and tungsten, and oxides of tantalum, gold, platinum, and tungsten.

11. A settable, magnetically guideable embolic material for occluding vascular defects, the material comprising:
between about 8 weight percent and about 12 weight percent prolamine
between about 15 weight percent and about 50 weight percent ethanol
between about 35 weight percent and about 45 weight percent of a magnetically responsive material; and
between about 25 and about 35 percent of a radio-opaque material.

12. The embolic material according to claim 11 further comprising polyvinyl acetate as a binder.

13. The embolic material according to claim 11 wherein the magnetically responsive material comprises Fe₃O₄.

14. The embolic material according to claim 14 comprising between about 35 weight percent and about 45 weight percent Fe₃O₄.

15. The embolic material according to claim 11 wherein the radio-opaque material comprises at least one of tantalum, gold, platinum, and tungsten, and oxides of tantalum, gold, platinum, and tungsten.

16. The material according to claim 11 wherein the ethanol is present as an aqueous solution of between about 70% and about 95% ethanol.

17. A settable, magnetically guideable embolic material for occluding vascular defects, the material comprising:
between about 8 weight percent and about 12 weight percent prolamine
between about 25 weight percent and about 45 weight percent of an aqueous solution of between about 70 weight percent and 95 weight percent ethanol;
between about 35 weight percent and about 45 weight percent of Fe₃0₄; and
between about 25 and about 35 percent of at least one radio-opaque material selected from the group comprising tantalum, gold, platinum, and tungsten, and oxides of tantalum, gold, platinum, and tungsten.

18. The embolic material according to claim 17 further comprising polyvinyl acetate as a binder.
